(19) United States
(12) Patent Application Publication
(10) Pub. No.: US 2014/0187998 A1
(43) Pub. Date: Jul. 3, 2014

(54) DEVICES AND TREATMENT METHODS FOR VASCULAR EYE DISEASES

(71) Applicants: Richard Lang, Indian Hill, OH (US); David Copenhagen, Oakland, CA (US)

(72) Inventors: Richard Lang, Indian Hill, OH (US); David Copenhagen, Oakland, CA (US)

(73) Assignees: THE REGENTS OF THE UNIVERSITY OF CALIFORNIA, Oakland, CA (US); CHILDREN'S HOSPITAL MEDICAL CENTER, Cincinnati, OH (US)

(21) Appl. No.: 14/126,402

(22) PCT Filed: Jun. 14, 2012
(86) PCT No.: PCT/US2012/042468

§ 371 Date: Mar. 11, 2014

Related U.S. Application Data
(60) Provisional application No. 61/496,937, filed on Jun. 14, 2011.

(51) Int. Cl. A61N 5/06; A61B 3/00

(52) U.S. Cl. CPC A61N 5/0613; A61B 3/0008
USPC 600/558; 607/88; 607/91

(57) ABSTRACT

A method of treating ROP, the method comprising providing a light source emitting light with a wavelength of about 490 nra, exposing an infant's eye to the light, and monitoring the vascularization in the infant's eye.

### FIELD OF THE INVENTION

This invention generally relates to devices and methods of treatment of vascular eye diseases. More particularly, this invention relates to devices and methods of treatment of vascular eye diseases of fetuses and infants.

### BACKGROUND

The embryonic eye develops with the aid of vascular networks that occupy the spaces between optical components. Evolution has provided mechanisms for regression of these vessels by the time of eyelid opening to ensure that refracted light can transit to the retina where high resolution images are formed. Without the regression of the vessels, vision is impaired. The lack of regression is known as retinopathy of prematurity (ROP).

Premature babies are often treated with high levels of oxygen, but the high levels of oxygen can lead to ROP. Careful control of the oxygen is required, and although reducing the oxygen level reduces the incidence of ROP, high levels of oxygen are needed to maintain the health of the premature baby. The risk for ROP increases as the severity of the prematurity.

The treatment of ocular disorders and conditions, including ocular vascular disorders and conditions, of new-borns and infants, can be treated with light emitted toward the eyes, but can be complicated by other medical treatment that the new-born receives which may include other light treatments (jaundice), oxygen (incubation) and intravenous therapy. Minimizing the number of procedures performed on the infant at any one time reduces the time and extra attention that medical staff must expend.

### SUMMARY

This invention relates to a method of treating ROP, the method comprising providing a light source emitting light with a wavelength of about 490 nm, exposing an infant's eye to the light, and monitoring the vascularization in the infant's eye.

This invention also relates to a method of treating ROP, the method comprising providing a light source emitting light with a wavelength of between about 465 and 515 nm, exposing an infant's eye to the light, and monitoring the vascularization in the infant's eye.

This invention relates to a method of treating ROP, the method comprising providing a light source emitting light with a wavelength of between about 465 and 515 nm, exposing an eye to the light, and monitoring the vascularization in the eye.

This invention also relates to a method for in utero light therapy comprising assessing a risk of a premature birth, determining a course of treatment to prevent ROP, providing a light source emitting light with a wavelength of between about 465 and 515 nm, and securing the light source to a body, wherein the light is directed toward a fetus in the body.

This invention also relates to a method for in utero light therapy comprising assessing a risk of a premature birth, determining a course of treatment to prevent ROP, providing a light source emitting light with a wavelength of about 490 nm, and securing the light source to a body, wherein the light is directed toward a fetus in the body.

This invention also relates to a method of treating a vascular disease comprising providing a light source emitting light having a wavelength of between about 450 and 530 nm, exposing the vascularly diseased area to the light, and monitoring treatment of the vascular disease against a standard.

This invention also relates to a device for providing in utero light therapy comprising a light source, wherein the light source provides light having a wavelength of between about 465 and 515 nm, and a securing device for securing the light source to a wearer's body, wherein the light is directed toward the body and wherein the light source is not directed at the eyes of the wearer.

This invention also relates to a device for providing in utero light therapy comprising a light source, wherein the light source provides light having a wavelength of about 490 nm, and a securing device for securing the light source to a wearer's body, wherein the light is directed toward the body and wherein the light source is not directed at the eyes of the wearer.

This invention also relates to a device for providing in utero light therapy comprising a light source, wherein the light source provides light having a wavelength of about 490 nm, and a securing device for securing the light source to a wearer's body, wherein the light is directed toward the body and wherein the light source is not directed at the eyes of the wearer, wherein 85% of the light provided by the light source has a wavelength between 460 nm and 520 nm.

This invention also relates to a device for treatment of ROP comprising an incubator with a lid, and a light source positioned above the lid, wherein the light provided by the light source has a wavelength of about 490 nm.

This invention also relates to a device for treatment of ROP comprising an incubator with a lid, and a light source positioned above the lid, wherein the light provided by the light source has a wavelength of between about 465 and 515 nm.

This invention also relates to a device for treatment of ROP comprising an incubator with a lid, and a light filtering device associated with the lid, wherein the light filtering device allows passage of only light with a wavelength of about 490 nm.

This invention also relates to a device for treatment of ROP comprising an incubator with a lid, and a light filtering device associated with the lid, wherein the light filtering device allows passage of only light with a wavelength of between about 465 and 515 nm.

This invention also relates to a method of creating a causative stimulus for organism development comprising providing a light source, regulating the light source, and monitoring the organism development.

This invention relates to a method of treating ROP, the method comprising providing a light source emitting light with a wavelength of about the excitation wavelength of melanopsin, exposing an eye to the light, and monitoring the vasculature in the eye.

This invention also relates to a method for in utero light therapy comprising assessing a risk of a premature birth, determining a course of treatment to prevent ROP, providing a light source emitting light with a wavelength of between about 465 and 515 nm, and securing the light source to the exterior surface of a wearer, wherein the light is directed toward a fetus in the body.

This invention also relates to a method for in utero light therapy comprising determining a course of treatment to prevent ROP, providing a light source emitting light with a wavelength of about the excitation wavelength of melanopsin, and securing the light source to a body, wherein the light is directed toward a fetus in the body.

This invention also relates to a method of treating a vascular disease comprising providing a light source emitting light having a wavelength of about the excitation wavelength of melanopsin, exposing the vascularly diseased area to the light, and monitoring treatment of the vascular disease against a standard.

This invention also relates to a device for providing light therapy comprising a light source, wherein the light source provides light having a wavelength of between about 465 and 515 nm, and a securing device for securing the light source to a wearer's body, wherein the light is directed toward the body and wherein the light source is not directed at the eyes of the wearer.

This invention also relates to a device for providing light therapy comprising a light source, wherein the light source provides light having a wavelength of about 490 nm, and a securing device for securing the light source to a wearer's body, wherein the light is directed toward the body and wherein the light source is not directed at the eyes of the wearer.

### BRIEF DESCRIPTION OF THE DRAWINGS

FIG. 1 is a perspective view of an embodiment of a light treatment device of the invention.

FIG. 2 is a perspective view an embodiment of another light treatment device with a remote controller of the invention.

FIG. 3 is a drawing of goggles of the invention with a light source.

FIG. 4 is a drawing of goggles of the invention with a filter.

FIG. 5 is a perspective view of a head-box of the invention.

FIG. 6 is a perspective view of an incubator of the invention.

FIG. 7 is a graph showing a mouse embryo's responsiveness to light.

FIG. 8 is a graph showing measurable light in the uterus for light transmitted through the body wall and with the body wall removed.

### DETAILED DESCRIPTION OF THE DRAWINGS

Research has shown that the hyaloid vessels, transiently residing between the lens and retina, regress, recede, or deteriorate in response to light. Dark-rearing mouse litters from late gestation produces pups with abnormally persistent hyaloid vessels 8 days after birth. This temporal response window eliminated involvement of rod and cone photoreceptors but implicated the melanopsin-expressing intrinsically photosensitive retinal ganglion cells (ipRGCs). Consistent with this, mice with a mutated melanopsin gene (Opn4) reared in a normal light cycle show persistent hyaloid vessels. Evidence shows that light stimulation via melanopsin suppresses retinal expression of vascular endothelial cell growth factor (VEGF) as a means of promoting apoptosis in hyaloid vascular endothelial cells (VECs). This is an example of a developmental process in which light stimulation results in a major change in vascular architecture.

Incorporated into the present application, is a manuscript authored jointly by the inventor(s) and other authors, describing the work conceived and put into practice by the inventor(s), which is entitled “Light Regulated Vascular Regression.” Additionally, the references and disclosures listed at the end of the manuscript are also incorporated by reference in their entirety.

Vascular Development Relating to Fetal and Infant Exposure to Light

The excitation wavelength of melanopsin is about 490 nm. By targeting a bandwidth centered around the excitation wavelength of melanopsin, optimum excitation of the melanospin, and thereby hyaloid vessel suppression or regression, may be achieved.

Research has also demonstrated fetal light responsiveness in rodents, primates, and humans. A researcher pregnant mouse dams were placed in the dark at different stages of late gestation (E16-17, E17-E18 or after E18) and then determined whether the hyaloid vessels were persistent at postnatal day 8 (P8). There is a dose-response where the hyaloids vessels were progressively more persistent with an earlier dark-rearing start. In particular, if dark-rearing was started after E18 (the day of birth is usually E19) there was almost no effect. These data indicate the result that the critical light-response window for hyaloids regression is E16-E17. This shows that the mouse embryo is light responsive, because there is an in utero gestational time window. Thus, exposure to light, and in particular light in the 490 nm range, can be used to excite the melanopsin, and thereby suppress vascularization in both the retinal and the vitreous by activating the expression of sFlt1, a suppressor of blood vessels. Additionally, research has shown that the peak transmittance of light through the body wall to the uterus of pregnant rodents occurs at just less than 500 nm.

As a result of these findings, various devices and treatment methods may be implemented to effect light-dependant vascular development. For example, and without limiting uses or patent coverage by way of the examples provided herein, light, and in particular possibly light with a wavelength of about 490 nm, could be used in utero to cause regression of the hyaloid vessels in a fetus expected to be born prematurely. Additionally, light, and in particular possibly light with a wavelength of about 490 nm, could be used to treat premature infants by causing a regression of the hyaloid vessels. In addition to these examples, other light-regulated development may also be stimulated or suppressed by artificial light or by intentional exposure to natural light. Also, while the methods and devices described herein are described with reference to a fetus, infant, or pregnant mammal, those descriptions are not limiting and the methods and devices may be applied to other mammalians.

Various methods of exposing a fetus or an infant to light to promote regression or suppression of the hyaloid vessels may be used. And various devices to accomplish the methods may also be used.

Treatment of Fetus

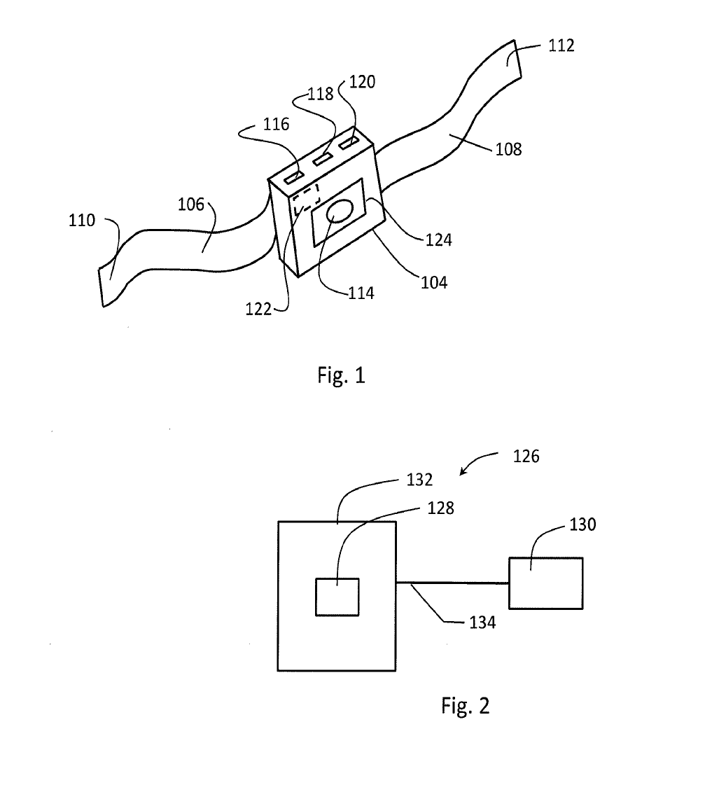

To treat a fetus, one would expose the fetus to a light outside of the mother. The light may be applied by way of a light source attached to a device that the mother wears or by way of enhanced natural light. One example of a fetal light treatment device is shown in FIG. 1. The fetal light treatment device 102 has a light source holder 104 connected to a first belt 106 and to a second belt 108. The belt 106 has a connecting device 110 and the second belt 108 has a corresponding connecting device 112. Instead of two belts, a single belt passing through or connected to the light source holder 104 may be used. The connecting devices, typically used for connecting the belt around the waist of a pregnant female, can be a belt, Velcro connector, or any other type of suitable connector. Additionally, for treatment of a body part instead of a fetus, the belt may be connected to another part of the body, such as a leg, arm, chest, or head. The light source holder 104 has a light source 114, a switch 116 for turning the light on, and a power source 122. The power source 122 can be a battery, or the power source may be standard AC electric. Additionally, the light source holder 104 may also have a transition timer 118, an intensity adjuster 120, and a pulse timer 119. For treatment, the light source holder 104 is placed so that the light source 114 is adjacent the treated organism.

The light source 114 can be various types of light sources, such as a LED, a light emitting capacitor (LEC), fluorescent light, or other type of light source. For treatment and/or prevention of ROP in utero, a light with a wavelength of about 490 nm would be desired. Treatment light with a 490 nm wavelength could be provided by a variety of methods. For treatment of ocular vascular conditions, including ROP, a method of the invention uses a light source that provides a light with a wavelength of in the light wavelength range of 490 nm+50 nm, more particularly 490 nm+25 nm. While light of other wavelengths can also be emitted from the light source 114, typically 85% of the light would have wavelengths between 390 nm and 590 nm, more typically 85% of the light would have wavelengths between 400 nm and 580 nm, more typically 85% of the light would have wavelengths between 410 nm and 570 nm, more typically 85% of the light would have wavelengths between 420 nm and 560 nm, more typically 85% of the light would have wavelengths between 430 nm and 550 nm, more typically 85% of the light would have wavelengths between 440 nm and 540 nm, more typically 85% of the light would have wavelengths between 450 nm and 530 nm, more typically 85% of the light would have wavelengths between 460 nm and 520 nm, typically 85% of the light would have wavelengths between 470 nm and 510 nm, and most typically, 85% of the light would have wavelengths between 480 nm and 500 nm. Other diseases could be treated with other wavelengths of light.

Another way of provided a desired wavelength of light is to provide a light source 114 providing full spectrum light, and then providing a filter 124 in between the light source 114 and the treated organism. For treatment of ROP, the filter would primarily allow only certain wavelengths of light to pass. While other wavelengths of light would be allowed to pass to some extent, for treatment of ROP, typically 85% of the light passing through the filter would have wavelengths between 390 nm and 590 nm, more typically 85% of the light passing through the filter would have wavelengths between 400 nm and 580 nm, more typically 85% of the light passing through the filter would have wavelengths between 410 nm and 570 nm, more typically 85% of the light passing through the filter would have wavelengths between 420 nm and 560 nm, more typically 85% of the light passing through the filter would have wavelengths between 430 nm and 550 nm, more typically 85% of the light passing through the filter would have wavelengths between 440 nm and 540 nm, more typically 85% of the light passing through the filter would have wavelengths between 450 nm and 530 nm, more typically 85% of the light passing through the filter would have wavelengths between 460 nm and 520 nm, typically 85% of the light passing through the filter would have wavelengths between 470 nm and 510 nm, and most typically, 85% of the light passing through the filter would have wavelengths between 480 nm and 500 nm.

Varying the timing of the light applied can also improve treatment and battery life. Pulsing LED lights is known to extend battery life, and the pulse timer 118 may have various set points for pulsing the light at various frequencies, turning the light on and off for various lengths of time. The light could be pulsed at frequencies ranging from tens of seconds to hundreds or even thousands of cycles per second.

The therapy may also be improved by multiple light transitions per 24 hours. One method of mimicking the natural 24 cycle would be to have the light on for 12 hours and then to have the light off for 12 hours. Additions transitions per day may improve therapy. The light can be operated on a 20 hour cycle, with 10 hours of on time followed by 10 hours of off time, a 16 hour cycle, with 8 hours of on time followed by 8 hours of off time, a 12 hour cycle, with 6 hours of on time followed by 6 hours of off time, or an 8 hour cycle, with 4 hours of on time followed by 4 hours of off time. Cycles with less time may also be used. And cycles with non-equal periods of light and dark may also be used. For example, the light could be on for 10 hours followed by an off time of 6 hours. And ratios of light to dark (L/D) may be used to describe the light cycle. For example, the L/D could be 6/1, 5/1, 4/1, 3/1, 2/1, 1/1, 1/2, 1/3, 1/4, 1/5, 1/6, 4/5, 7/3, or any other combination of L/D ratios. The transition timer 119 may be used to set the proper light transition cycle.

The therapy may also be improved by adjusting the intensity of the light. While there is expected to be a lower threshold limit below which the light is ineffective and an upper limit above which there is no improved benefit (or even perhaps harm is caused), there is a range between the lower limit and the upper limit where the therapy is proportional to the light intensity. Depending on the severity of the condition, the light intensity may be adjusted to meet the treatment protocol for the condition. The intensity adjuster 120 on the light box 104 is used to adjust the light intensity.

Multiple combinations of pulses, transition times, intensity, and treatment duration may be used to optimize treatment. Depending on the severity of the condition, treatment may take from a day to several weeks or more.

Other methods of wearing the light source holder in addition to a belt may also be used. FIG. 2 shows another embodiment of a fetal treatment device. In FIG. 2 the fetal treatment device 126 is contains a memory device 128 that is programmed by a controller 130. The memory device 128 may also include a microprocessor. A light source holder 132 holds a light source (not shown). The light source can be various types of light sources, such as a LED, a light emitting capacitor (LEC), fluorescent light, or other type of light source. The controller 130 contains the apparatus needed to program the memory device 128 of the light source holder, including the ability to adjust intensity, pulses, transitions and to activate the light source holder. The controller 130 may be permanently or temporarily attached to the light source holder 132 by way of a communications cable 134. If the attachment is temporary, then the controller 130 is temporarily attached to the light source holder 132 while the controller 130 is used to program the memory device. Alternatively, instead of a communications cable 134, the controller may communicate with the light source holder wirelessly. The light source holder 132 could be attached to the skin with an adhesive or a double stick tape instead of a belt.

The types of devices described above can be used not only to treat or prevent fetus's with ROP, but also to treat other disorders that are light responsive. While different opsins may be excited by wavelengths other than 490 nm, the devices described above could be easily modified to provide the required wavelength of light for treatment of other disorders without falling outside the scope of this patent. For instance, this treatment with light of similar wavelengths to that described here or with light of different wavelengths, depending on the condition, could be used to treat other vascular disorders of the eye such as diabetic retinopathy and wet and dry type macular degeneration.

Treatment of an Infant Using Goggles

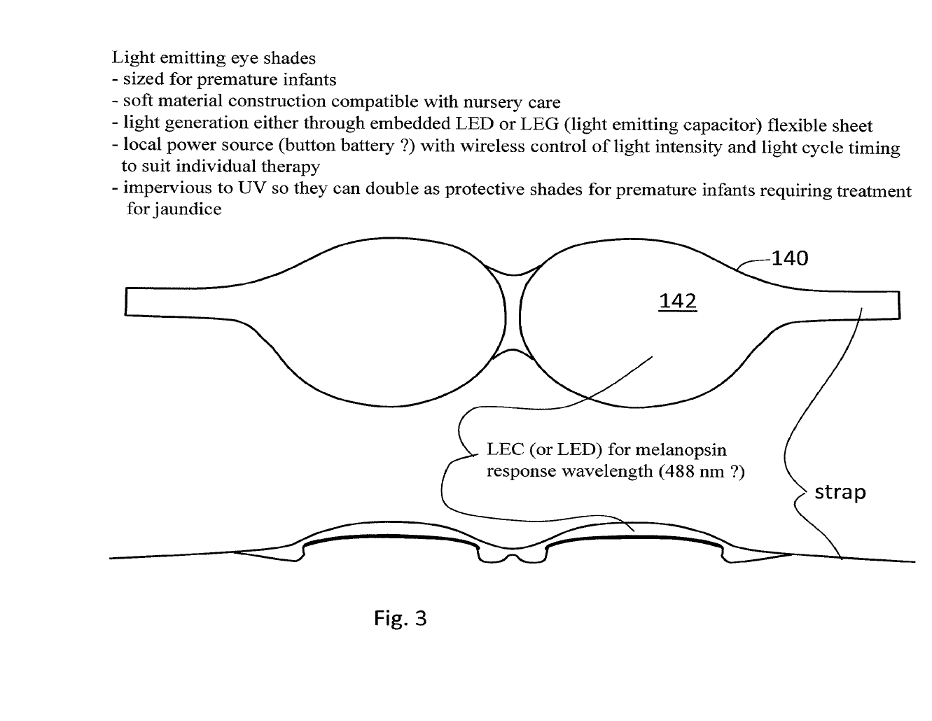

To treat a newborn, other devices can be used to provide the proper wavelength of light to the newborn. FIG. 3 shows one type of device that may be used to treat newborns. The device includes goggles 140 with a light source 142. The light source 142 can be a LED, LCD, or other type of light source. The goggles may be powered by a battery, such as a button battery, or they may be powered by standard plug-in AC electric. Typically, the light source has a wavelength of about 490 nm. While light of other wavelengths would also be emitted from the light source 114, typically 85% of the light would have wavelengths between 390 nm and 590 nm, more typically 85% of the light would have wavelengths between 400 nm and 580 nm, more typically 85% of the light would have wavelengths between 410 nm and 570 nm, more typically 85% of the light would have wavelengths between 420 nm and 560 nm, more typically 85% of the light would have wavelengths between 430 nm and 550 nm, more typically 85% of the light would have wavelengths between 440 nm and 540 nm, more typically 85% of the light would have wavelengths between 450 nm and 530 nm, more typically 85% of the light would have wavelengths between 460 nm and 520 nm, typically 85% of the light would have wavelengths between 470 nm and 510 nm, and most typically, 85% of the light would have wavelengths between 480 nm and 500 nm.

In FIG. 3 the goggles 140 may also is contain a memory device 144 that is programmed by a controller similar to that described above. The memory device 144 may also include a microprocessor. The controller 130 can be used to program the memory device 144 of the light source holder, including the ability to adjust intensity, pulses, transitions and to activate the light source holder. The controller may be permanently or temporarily attached to the goggles 140 by way of a communications cable. If the attachment is temporary, then the controller is temporarily attached to the light source holder 132 while the controller 130 is used to program the memory device. Alternatively, instead of a communications cable 134, the controller may communicate with the light source holder wirelessly. In addition to providing light, the goggles may be include a filter impervious to UV light so they can also be used as protective shades for premature infants requiring treatment for jaundice.

In a hospital nursery, multiple premature infants may require treatment with the goggles at the same time. In those cases, a master controller, with the functions of the controller described above, could be used to program multiple goggles. The master controller could be located in a central location and could wirelessly communicate with the multiple goggles.

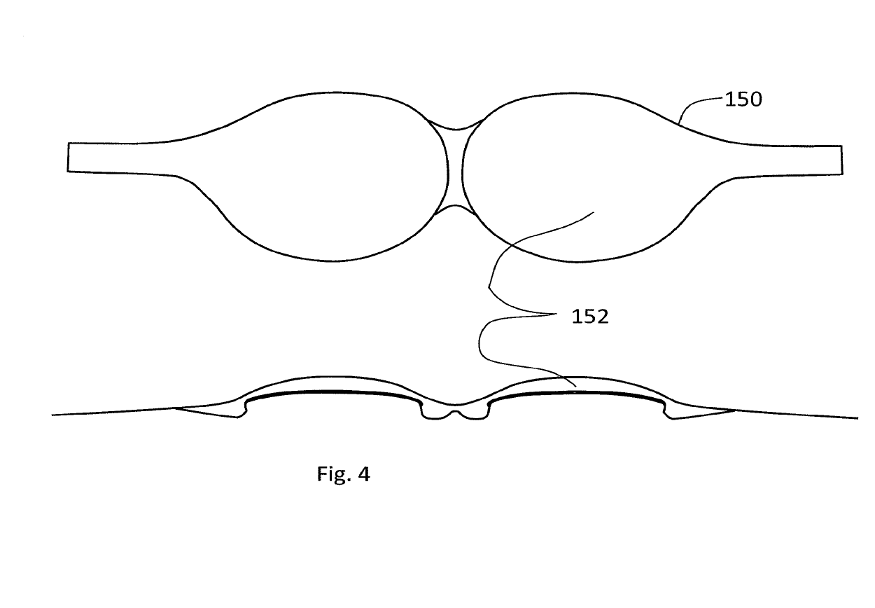

FIG. 4 shows another method of treating premature infants using goggles 150. The goggles 150 have a filter 152. For treatment of ROP, the filter 152 would primarily allow only certain wavelengths of light to pass. While other wavelengths of light would be allowed to pass to some extent, for treatment of ROP, typically 85% of the light passing through the filter would have wavelengths between 390 nm and 590 nm, more typically 85% of the light passing through the filter would have wavelengths between 400 nm and 580 nm, more typically 85% of the light passing through the filter would have wavelengths between 410 nm and 570 nm, more typically 85% of the light passing through the filter would have wavelengths between 420 nm and 560 nm, more typically 85% of the light passing through the filter would have wavelengths between 430 nm and 550 nm, more typically 85% of the light passing through the filter would have wavelengths between 440 nm and 540 mm, more typically 85% of the light passing through the filter would have wavelengths between 450 nm and 530 nm, more typically 85% of the light passing through the filter would have wavelengths between 460 nm and 520 nm, typically 85% of the light passing through the filter would have wavelengths between 470 nm and 510 nm, and most typically, 85% of the light passing through the filter would have wavelengths between 480 nm and 500 nm.

By limiting the wavelength of light reaching the infant's eyes, goggles 150 can be used with full spectrum light of an intensity that would otherwise be harmful to the infant. Thus, an additional full spectrum light source could be placed above the infant's bed to provide additional amounts of 490 nm light than would otherwise be available. Additionally, the filter not only provides for the treatment of ROP, but also blocks the UV rays that are used to treat jaundice.

Treatment of an Infant Using a Head-Box

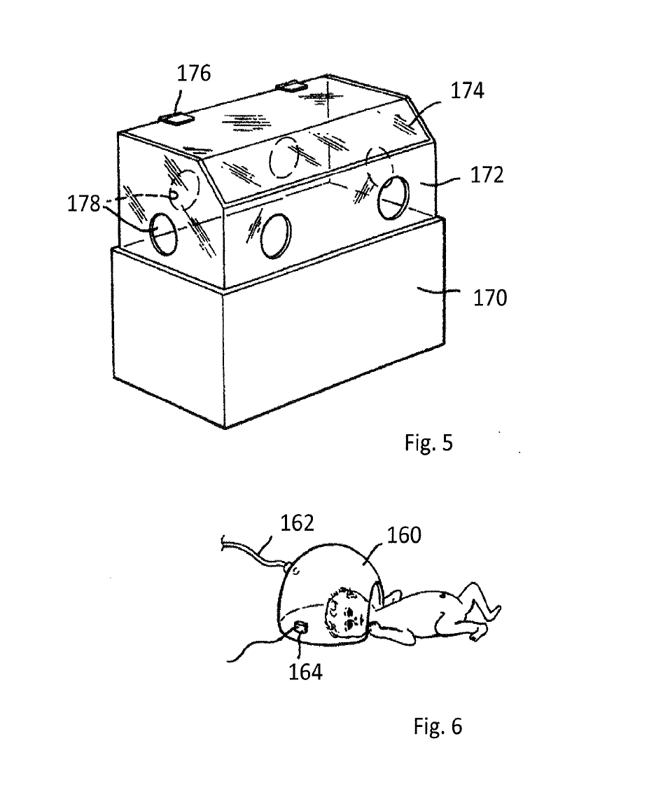

Another way to treat infants is shown in FIG. 5. A head-box is usually used in an incubator and placed over the infant's head to maintain a high local oxygen level. The head-box 160 has a humidified oxygen inlet 162 and an oxygen level sensor 164. When used to treat ROP, the head-box 160 can be made of a filtering material allowing only about 490 nm light to pass, or it can be a standard head-box treated with a filtering material allowing only about 490 nm light to pass. The typical wavelength of light that is allowed to pass is similar to those various wavelengths described for the filtering goggles. And as with the goggles, by limiting the wavelength of light reaching the infant's eyes, the head-box 160 can be used with full spectrum light of an intensity that would otherwise be harmful to the infant. Thus, an additional full spectrum light source could be placed above the infant's bed to provide additional amounts of 490 nm light than would otherwise be available.

Instead of using a head-box made with a filtering material or a standard head-box with applied filtering material, another way to treat is to utilize a light with 490 nm wavelength, with the typical wavelengths as described above, placed above the head-box. The light could be a LED light, a LEC light, an incandescent light, a fluorescent light, or any other type of light with the required wavelength. With a flat sheet-like profile, an LEC could be placed on top of the head-box for treatment.

Treatment of an Infant Using an Incubator

Another way to treat infants is shown in FIG. 6. FIG. 6 shows an incubator having a base 170, a hood 172, the hood having a lid 174 with a hinge 176. The incubator may also have climate control means for sensing conditions such as humidity, temperature, and oxygen concentration and maintaining them at desired levels. A baby is placed on a mattress located on the base 170 under the hood 172. The hood 172 has a plurality of access ports 178 spaced around its sides, each closed by a flexible sheet with a hole with an elasticized edge. When used to treat ROP, the hood 172 can be made of a filtering material allowing only about 490 nm light to pass, or it can be a standard hood treated with a filtering material allowing only about 490 nm light to pass. The typical wavelength of light that is allowed to pass is similar to those various wavelengths described for the filtering goggles. And as with the goggles, by limiting the wavelength of light reaching the infant's eyes, an incubator with a wavelength limiting hood can be used with full spectrum light of an intensity that would otherwise be harmful to the infant. Thus, an additional full spectrum light source could be placed above the infant's bed to provide additional amounts of 490 nm light than would otherwise be available.

Instead of using an incubator with a hood made with a filtering material or a standard hood with applied filtering material, another way to treat is to utilize a light with 490 nm wavelength, with the typical wavelengths as described above, placed above the incubator. The light could be a LED light, a LEC light, an incandescent light, a fluorescent light, or any other type of light with the required wavelength. With a flat sheet-like profile, an LEC could be placed on top of the lid of the incubator for treatment.

The methods and devices for treating premature infants described above are effective for the treatment of ROP whether the infant's eyes are open or closed, because of the demonstrated ability of light with a wavelength of about 490 nm to pass through the skin.

Other publications have described devices utilizing a light source or a means of filtering wavelengths of light, including: U.S. Pat. No. 5,336,248, Good et al, which describes a plastic, oxygenating incubator, wherein the plastic material is transparent only to red light (612 nm and above); US Publ 2006-0136018, Lack et al, which describes a pair of glasses with a means for supporting at least two LEDs in front of each pupil, to emit light in the wavelength range of 450-530 nm for the purpose of re-timing the human body clock for overcoming jet lag and other sleep disorders; U.S. Pat. No. 7,901,071, Kulas, which discloses eyewear with a controllable LED light that emits light in response to ambient light intensity or object proximity; U.S. Pat. No. 4,938,582, Leslie, which describes chromo therapeutic glasses having an opaque shade lenses and LED lights of various colors fitted to shine on the inside surface of the shade lenses; GB 2,196,442, Anderson (describes a stroboscopic LED light mounted onto swim goggles); US Publ 2009-0260633, Vreman, which describes eye shades that are selectively transparent, which allowing transmission of only 2-20% of all light with wavelength between 400-610 nm (yellow to blue); U.S. Pat. No. 6,511,175, Hay et al, which discloses eyewear having electrically-controllable LCD lens to selectively darken one lens or the other for treating amblyopia; U.S. Pat. No. 5,264,877, Hussey, which describes an electro-optical composite film that is cloudy/opaque without a current passing through the film, and transparent when a current is passed through, for treating lazy eye, etc; U.S. Pat. No. 7,942,524, Smith et al, which describes eyewear with independently-activated LED lights to provide light stimulus for treating unilateral neglect syndrome; U.S. Pat. No. 4,790,031, Duerer, which describes an eye shield for sun bathers that can include a sun-blocking material that blocks ultraviolet (UV) rays; and US Publ 2008/0039906, Huang, which discusses a light therapeutic device; the disclosures of which are incorporated by reference in their entirety.

Methods of Treatment

One protocol for treatment of a high risk ROP patient would include an assessment of the pregnant female to determine whether premature birth is a risk and at what stage in the pregnancy the premature birth might occur. Balancing the fact that the hyaloid vessels are necessary for eye development and early treatment in utero may prevent proper eye development against the risk of ROP, treatment with one of the devices for fetal treatment described above could be initiated. Depending on the estimated prematurity of the birth, the treatment start time and duration and the transition times, intensity, and pulsing of the light can be optimized for proper treatment.

One protocol for treatment of a premature infant with ROP includes the diagnosis of the ROP, assessment of the estimated time of treatment necessary to treat the ROP, and implementation of ROP treatment utilizing one of the devices described above. During the treatment, medical professionals can monitor the vascularization of the eye to assess the efficacy of the treatment, adjust the treatment duration, transition times, and intensity and pulsing of the light as necessary, and cease treatment when the ROP is deemed sufficiently cured. Depending on the prematurity of the birth, the treatment duration, transition times, and intensity, and pulsing of the light can be optimized for proper treatment.

The typical ranges of the light wavelengths described in paragraph [0034], the typical light transitions described in paragraph [0037], the pulsing of the light described in paragraph [0036], and the use of a controller to control these factors and others such as intensity, are applicable to the other devices and methods described herein.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention is therefore not limited to the specific details, representative apparatus and method, and illustrated examples shown and described. Accordingly, departures may be made from such details without departing from the scope or spirit of the invention.

### EXPERIMENTAL RESULTS

### The Critical Light-Response Window for Hyaloid Regression in the Mouse is Prior to Birth

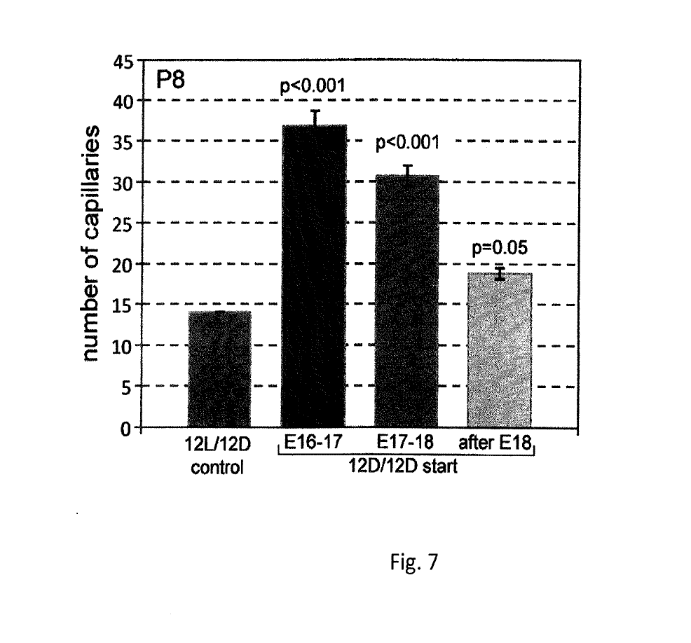

In this experiment, the results are which are shown in FIG. 7, experimenters placed pregnant dams in the dark at different stages of late gestation (E16-17, E17-E18 or after E18) and determined whether the hyaloid vessels were persistent at postnatal day 8 (P8). There is a dose-response where the hyaloids vessels were progressively more persistent with an earlier dark-rearing start. In particular, if dark-rearing was started after E18 (the day of birth is usually E19) there was almost no effect. These data indicate the result that the light-response window for hyaloid regression is E16-17. Since this is an in utero, gestational time-window, there is a very clear implication that the mouse embryo is light responsive. Since melanopsin is already expressed in the retina at this stage of embryonic development, these data are entirely consistent with hyaloid persistence in the Opn4 mutant mouse.

The possibility of direct fetal light-responsiveness in rodents, primates and humans is strongly supported by a small number of publications, which make the following critical points:

Light of the Appropriate Wavelength to Stimulate Melanopsin Penetrates to the Uterus in Pregnant Rodents.

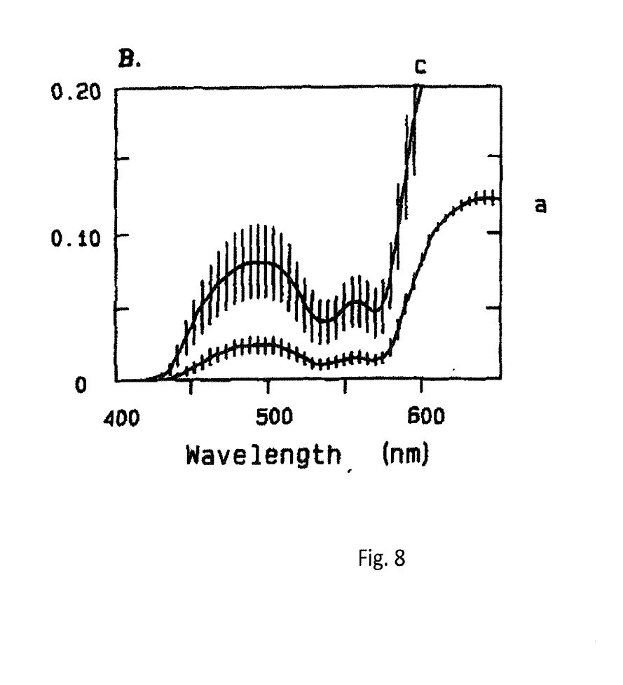

FIG. 8, excerpted from (Jacques, S. L., Weaver, D. R. and Reppert, S. M. (1987). Penetration of light into the uterus of pregnant animals. Photochem Photobiol 45, 637-41) shows measurable light levels in the uterus transmitted through the body wall (a) or with the body wall removed (c). Of interest is the observation that some light does get through the intact body wall and the peak transmittance at just less than 500 nm corresponds very closely to the 480 nm excitation wavelength of melanopsin. This study was performed with rats and guinea-pigs.

Fetal Rodents are Directly Light-Responsive.

In support of the notion that fetal mammals are directly light-responsive, pregnant dams were “enucleated” (their eyes were removed) and the light-dependent uptake of radiolabeled glucose in the suprachiasmaic nucleus (SCN, the circadian pacemaker) was assessed in both dam and fetus. (Weaver, D. R. and Reppert, S. M. (1989). Direct in utero perception of light by the mammalian fetus. Brain Res Dev Brain Res 47, 151-5). A light response was observed only in the fetus. This response is known to be melanopsin-dependent.

The Circadian System of Premature Infant Primates is Light Responsive.

Hao and Rivkees showed that non-human primates show a light-responsive circadian oscillator at a stage of development equivalent to 24 weeks of human fetal gestation. (Hao, H. and Rivkees, S. A. (1999). The biological clock of very premature primate infants is responsive to light. Proc Natl Acad Sci USA 96, 2426-9.) This response is melanopsin-dependent. This paper actually makes very interesting reading as the authors comment very pointedly that little thought is given to the light conditions in hospital nurseries. That was 1999, but is still the case now.

The Human Fetus is Responsive to Light Transmitted Through the Body Wall.

Using magnetoencephalography it has been shown that the human fetus responds to light flashes delivered to the abdomen of the pregnant mother. (Eswaran, H., Lowery, C. L., Wilson, J. D., Murphy, P. and Preissl, H. (2004). Functional development of the visual system in human fetus using magnetoencephalography. Exp Neurol 190 Suppl 1, S52-8). Interestingly, the amount of light delivered was 8,800 lux, less than 10% of the light intensity represented by a sunny day (100,000 lux). So again, the fetus is responsive to quite low light levels and this is consistent with the expression of melanopsin in the human fetus after 8 weeks of gestation (Tarttelin, E. E., Bellingham, J., Bibb, L. C., Foster, R. G., Hankins, M. W., Gregory-Evans, K., Gregory-Evans, C. Y., Wells, D. J. and Lucas, R. J. (2003). Expression of opsin genes early in ocular development of humans and mice. Exp Eye Res 76, 393-6).

### Light Regulated Vascular Regression

### Sujata Rao^{1,2}, Christina Chun³, Jieqing Fan^{1,2}, Napoleone Ferrara⁴, David Copenhagen³*, and Richard A. Lang^{1,2}*

1. The Visual Systems Group, Divisions of Pediatric Ophthalmology and Developmental Biology, Cincinnati Children's Hospital Medical Center, Cincinnati, Ohio 45229, USA
2. Department of Ophthalmology, University of Cincinnati, Cincinnati, Ohio 45229, USA
3. Departments of Ophthalmology and Physiology, University of California, San Francisco, San Francisco, Calif. 94143.
4. Genentech Inc., 1 DNA Way, South San Francisco, Calif. 94080, USA

### *Corresponding Authors:

Richard A. Lang, Division of Pediatric Ophthalmology, Cincinnati Children's Hospital Medical Center, 3333 Burnet Avenue, Cincinnati, Ohio 45229, Tel: 513-636-2700 (Office), 513-803-2230 (Assistant), Fax: 513-636-4317. Email: Richard.Lang@cchmc.org
David R. Copenhagen, Departments of Ophthalmology and Physiology, University of California, San Francisco, San Francisco, Calif. 94143. Tel: 415-476-2527 or 415-476-3171. Fax: 415-476-6289. Email: cope@phy.ucsf.edu
   The embryonic eye develops with the aid of vascular networks that occupy the spaces between optical components. Evolution has provided mechanisms for regression of these vessels by the time of eyelid opening to ensure that refracted light can transit to the retina where high resolution images are formed. Here we show that the hyaloid vessels, transiently residing between lens and retina, regress in response to light. Dark-rearing mouse litters from late gestation produces pups with abnormally persistent hyaloid vessels 8 days after birth. This temporal response window eliminated involvement of rod and cone photoreceptors but implicated the melanopsin-expressing intrinsically photosensitive retinal ganglion cells (ipRGCs). Consistent with this, mice with a mutated melanopsin gene (Opn4) reared in a normal light cycle show persistent hyaloid vessels. Furthermore, we provide evidence that light stimulation via melanopsin suppresses retinal expression of vascular endothelial cell growth factor (VEGF) as a means of promoting apoptosis in hyaloid VECs. This is an unusual example of a developmental process in which light stimulation results in a major change in vascular architecture.

The pupillary membrane, tunica vasculosa lentis and hyaloid vessels of the eye are anatomically distinct but connected vascular networks that undergo scheduled involution¹. Regression of these vessel networks involves BMP4 for the pupillary membrane² and collagen XVIII³, macrophage production of Wnt7b⁴ and angiopoietin⁵ signalling pathways for the hyaloid vessels. Despite progress in understanding mechanisms of scheduled vascular regression, the causative signal has remained elusive.

Prompted by the strong rationale for light-triggered developmental events in the eye⁶, and by the recent recognition that newborn mice are light-responsive⁷, we tested whether the hyaloid vessels changed their regression timing under conditions of modified light exposure, The hyaloid vessels reside between the lens and retina and normally regress progressively between 1 and 9 days after birth (P1-P9)¹. When pregnant dams were placed in the dark during late gestation at E16-17 and their pups raised in the dark thereafter, the hyaloid vessels were abnormally persistent at P8 (FIG. 1a, b) compared with controls raised in normal lighting conditions (hereafter, we refer to normal lighting conditions—12 hours light, 12 hours dark, as LD and constant darkness as DD). A time-course of hyaloid capillary number (FIG. 1b) reveals that newborn (P1) dark-reared pups show no difference in hyaloid vessel number and that a significant failure of hyaloid regression only becomes evident at P8 (FIG. 1a, b). This shows that dark-rearing from late gestation does not effect formation of the hyaloid vessels, but only the process of regression. The degree of hyaloid persistence with dark-rearing was typical of the previously characterized Ang2 and Lrp5 mutants^{4,5} and like these, showed a reduced level of vascular endothelial cell (VEC) apoptosis (FIG. 1c) that was the likely downstream cause of regression failure.

Melanopsin is the only opsin known to function in the mouse eye before P10⁸. Light activation of melanopsin can mediate the pupillary reflex⁹ and photoentrainment of the circadian cycle in adults^{10,11}. In neonates, it mediated negative phototaxis⁷. Since melanopsin is functional before P10, it was a good candidate to mediate light-dependent hyaloid regression. To test this possibility, we assessed hyaloid vessel regression in the gene-targeted Opn4 mutant mice^{10,11}. In contrast with control littermates Opn4 homozygotes showed a robust persistence of the hyaloid vessels at P8 (FIG. 2a, b). In this analysis, mutant pups were generated by crossing heterozygous parents. Hyaloid vessel assessment was then performed in sets of littermates co-reared in LD conditions where Opn4^{+/+} littermates served as an internal control. This experimental design effectively excludes the possibility that a melanopsin-dependent defect in the dam is responsible for hyaloid persistence.

Melanopsin expressing ipRGCs^{12,13} are a subset of RGCs that can be located in the P5 retina with immunofluorescence labeling (FIG. 2c, d). Double labeling for vasculature (FIG. 2c, green) and melanopsin (FIG. 2c, d, red) shows that melanopsin is expressed in the superficial layers of the retina immediately adjacent to the vitreous in which the hyaloid vessels reside. Thus, melanopsin is expressed at the right time and in a place consistent with the possibility of light-regulated hyaloid regression.

VEGF is a potent signal for VEC survival¹⁴ that is expressed in the superficial layers of the mouse postnatal retina¹⁵ and is present in the vitreous of the rodent¹⁶ and human¹⁷ eye. We hypothesized that light-dependent hyaloid regression might be explained by modulation of VEGF, or of its naturally occurring inhibitory receptor Flt¹⁸⁻²¹. Consistent with the latter hypothesis, and with an Flt1 expression phase from P1 to P7 in superficial retina¹⁵, germ line loss of function Flt1 heterozygotes showed hyaloid persistence (FIG. 3a, b). To determine whether the lens or retina was a source of hyaloid-regulating Flt1, we performed conditional deletion of the Flt^{fl} allele²² using either the lens-specific Le-cre²³ or the retina-specific Chx10-cre driver²⁴. Deletion of Flt1 in the lens had no effect on hyaloid regression (data not shown) but retinal deletion resulted in hyaloid persistence (FIG. 3c, d). A time-course of hyaloid regression in Chx10-cre control and Chx10-cre; Flt1^{fl/fl} mice showed that there was no significant change in the development of hyaloid vessels at P3, but that hyaloid persistence was evident in the conditional mutant mice by P8 (FIG. 3d). Combined, these data indicate that retinal Flt1 is required for hyaloid vessel regression.

To determine whether the soluble form of Flt1 (sFt1¹⁸) might be a light-regulated modulator of hyaloid regression, we examined the level of sFlt1 in vitreous over the P3-P8 time-course using immunoblotting (FIG. 3e). Two experiments with distinct detection sensitivities nonetheless showed that in the vitreous of wild-type mice, the level of sFlt1 declines modestly during hyaloid regression. This is opposite to expectation; if a change in the level of sFt was a key trigger of hyaloid regression it should increase over this time-course. We then quantified by QPCR the level of retinal Flt1 transcript in dark-reared or Opn4 mutant mice (FIG. 3f) but could detect no statistically significant change. ELISA detection of vitreous sFlt1 showed that in the Opn4 mutant, there was no significant change (FIG. 3g, light blue bar) thus indicating that the hyaloid persistence of the Opn4 mutant (FIG. 2a) was not a consequence of decreased levels of sFlt1. P5 vitreous from dark-reared pups did show a modest (2-fold) modulation in sFlt1 (FIG. 3g, dark blue bar) however, the level went up, not down, as would be anticipated if sFlt1 was a light-dependent regulator of hyaloid regression. Combined, these data provide compelling evidence that although retinal Flt1 can clearly influence hyaloid vessel regression, it is not the mediator of light and melanopsin-dependent regression.

We then considered the alternative hypothesis that expression of retinal VEGF was light-regulated. Deletion of a VEGF conditional allele from the lens results in a failure of development of the tunica vasculosa lentis (the capillaries adhered to the lens capsule) but has no effect on development of the hyaloid vessels adjacent to the retina²⁵. This indicates that VEGF has a local angiogenic action. By extension, it was possible that reduction of retinal VEGF could result in hyaloid regression. To address this question, we deleted the VEGF^{fl} conditional allele (ref 22) in the retina using Chx10-cre and showed that the level of VEGF immunoeactivity was successively reduced in Chx10-cre; VEGF and Chx10-cre; VEGF^{fl/fl} retina (FIG. S1).

When the Chx10-cre retinal driver²⁴ was used to generate a homozygous VEGF^{fl} we found that the hyaloid vessels failed to develop (FIG. 4a, yellow arrows) but that the tunica vasculosa lentis was unaffected (FIG. 4a, white arrows). This showed that the hyaloid vessels were dependent on local retinal VEGF for their formation but did not implicate VEGF in hyaloid regression. As an alternative, we performed heterozygous, Chx10-cre mediated VEGF^{fl} deletion. We noticed that the control, VEGF^{fl/+} genotype consistently produced hyaloid vessel structures that were denser than normal at P8 (FIG. 4b, c). Though we do not have a detailed explanation for this, we cannot attribute this to genetic background because the increased hyaloid density tracks only with the VEGF^{fl/+} genotype. It is more likely that the VEGF^{fl} allele design produces a mild over-expression of VEGF that has a consequence for the hyaloid vessels. Regardless, when the VEGFA^{fl} allele was deleted to heterozygosity with Chx10-cre, the hyaloid vessels showed reduced density at P8 (FIG. 4b, c) and this argues that the hyaloid vessels are sensitive to VEGF withdrawal during the normal phase of regression.

An immunoblot for vitreous VEGF over the P1-P8 time-course revealed that VEGF levels were reduced at P5 but have risen again by P8 (FIG. 4d). The mobility of the detected band is consistent with the VEGF 164 isoform. When three different VEGF time-course immunoblots were quantified, the P5 VEGF signal was about 5-fold reduced compared with P1 (FIG. 4d). A low level of VEGF at P5 is consistent with the idea that it is a key regulator of hyalold regression because P5 is the time when there are peak levels of VEC apoptosis⁴. We next determined, using dark rearing and the Opn4 mutant mice, whether actual or functional darkness resulted in a modulation of vitreous VEGF. In immunoblots (FIG. 4e) we consistently observed that VEGF levels were increased in darkness or when light responsiveness was compromised. Six independent immunoblot experiments are shown, each with shorter and longer film exposures. At P1, the Opn4 mutant mice show a higher level of VEGF signal (FIG. 4e). In two independent assessments of vitreous VEGF levels in P5 Opn4 mutant mice, the detection sensitivity varied, but in both cases there was a relative increase in VEGF levels (FIG. 4e). In vitreous from dark-reared pups, we also observed an increase in vitreous VEGF levels at P1 and in two separate assessments of the P5 time-point (FIG. 4e). Furthermore, an ELISA-based assessment of VEGF in the P5 vitreous shows that whether pups were dark-reared or mutated in Opn4, the levels of VEGF were about 7-fold fold higher than in the control (FIG. 4f). A substantial fold increase in VEGF in the vitreous of dark-reared and Opn4 mutants was reflected in similar fold increases in the level of retinal VEGF mRNA as indicated by QPCR (FIG. 4g). This was consistent with the genetic analysis where deletion of retinal VEGF^{fl} resulted in hyaloid regression (FIG. 4a). Finally, to gain some understanding of the net change in VEGF activity in dark-reared and Opn4 mutant mice, we expressed their levels as a VEGF/sFlt1 molar ratio based on ELISA quantifications (FIG. 3g, 4f). This showed that the P5 vitreous of a control mouse has a molar ratio of about 6 while that of dark-reared is 78 and Opn4 is 155. This indicates that there is a very robust increase in the potential activity of VEGF. Given the VEGF dependence of the hyaloid vessels, VEGF abundance is an explanation for persistence. Combined, these data suggest that normally, a melanopsin-mediated, light-dependent response suppresses VEGF expression. In turn, we propose that suppression of VEGF levels is a component of the normal hyaloid vessel regression program.

These studies identify light as a causative stimulus for scheduled vascular regression in the eye. This is surprising because is has not been shown previously that light can trigger major changes in vascular architecture. The timing of hyaloid regression precedes photoreceptor function and thus is the perfect preparation for the image-forming capability of the eye. Given the influence that light stimulation has on the physiology of all organisms, we suspect that other examples of light-regulated development will emerge. Certainly, the family of non-photoreceptor opsins is large and shows wide expression, but little is currently know about their function.

Existing studies have shown that regression of the hyaloid vessels is dependent on collagen XVIII³, the macrophage Wnt ligand Wnt7b⁴ and the context-dependent angiopoietin pathway antagonist angiopoietin2⁵. However, given evidence that VEGF was present in the vitreous^{16,17}, it was unclear why the survival stimulating effects of this potent factor did not override pro-apoptotic stimuli. The current analysis argues that a reduction in vitreous VEGF level, most likely quite transient, is a critical contributor to the regression program. Reduced levels of VEGF are likely to promote hyaloid regression by reducing the threshold sensitivity for triggering apoptosis in response to pro-apoptotic stimuli that include Angiopoietin2⁶ and Wnt7b⁴.

One implication of this analysis is that light may be a useful medium to treat the perinatal ocular disease retinopathy of prematurity (ROP)²⁶. This defect arises when there is a rebound vascular overgrowth in the retina after the newborn has transitioned to the high oxygen tension of the external environment. ROP is known to be VEGF mediated and can be treated successfully with intra-vitreal injections of anti-VEGF antibodies²⁷. Here we show that light stimulation suppresses VEGF expression by the retina and so it may be that the attempt to treat ROP by lowering the light levels for premature infants^{28,29} was counterproductive. Based on the developmental pathway described here, increased, not decreased light exposure would be more likely to reduce the risk of ROP because the level of VEGFA expression might be suppressed.

### Methods Summary

All animals used for dark rearing were from the C57BL/6 background. To asses hyaloid vessel persistence from the dark reared animals, pregnant dams were placed in the dark at indicated times and the eyes were harvested from the pups. ELISA's and western blots for sFLT1 and VEGF were performed on vitreous harvested from animals reared in the dark and melanopsin nulls. To assay for differences in transcript levels of VEGFA and sFLT1 QPCR's were performed using the entire retina.

### Methods

### Quantification of Hyaloid Vasculature and Melanopsin Immunofluorescence

Hyaloid Vessels were harvested and stained with Hoechst as well as for TUNEL as described⁴. Retinas were labeled for melanopsin as reported (except that Alexa Fluor 488 isolectin GS-IB₄ (Invitrogen) was used to label retinal vessels¹¹.

### Isolation of Vitreous

Vitreous was harvested from dark reared pups in a dark room, using an infrared light source. Eyes from P1 and P5 pups were washed twice in sterile ice cold PBS, Excess PBS was blotted using a kimwipe, a small slit was made through the retina and vitreous harvested.

### Protein Analysis

ELISA was performed on the vitreous using the sVEGFR1 and VEGF Quantikine kits (R&D). Immunoblots were probed with a unique C-terminal antibody for sFLT1²¹ and VEGFA³⁰. Quantification was performed using ImageJ.

### RNA Isolation and RT-PCR

RNA was isolated using RNeasy (Qiagen). QPCR was performed with QuantiTect SYBR green (Qiagen). Primers were as follows:

sFLT1
5′-ATGCGCTGCAGAGCCAGGAAC-3′,
5′-GGTACAATCATTCCTCCTGC-3′.
VEGFA
5′-GACAGAACAAAGCCAGA-3′,
5′-CACCGCCITGGCTTGTCAC-3′.

### Statistics

All statistical tests used are stated in the figure legends. In analyzing QPCR data, the p values refer to a comparison of the ΔΔcT values.

### Animals

Breeding and genotyping of VEGF^{fl}, Flt^{fl} (ref 22), chx10-cre²⁴ Opn4^{-/-} (ref 10) was performed as previously described. All animal experimentation was carried out using protocols approved by the institutional Animal Care and Use Committee,

### REFERENCES

¹ Ito, M. & Yoshioka, M., Regression of the hyaloid vessels and pupillary membrane of the mouse. Anatomy and Embryology 200, 403-411 (1999).
² Kiyono, M. & Shibuya, M., Bone morphogenetic protein 4 mediates apoptosis of capillary endothelial cells during rat pupillary membrane regression. Mol Cell Biol 23 (13), 4627-4636 (2003).
³ Fukai, N. et al., Lack of collagen XVIII/endostatin results in eye abnormalities. Embo J 21 (7), 1535-1544 (2002).
Lobov, I. B. et al., WNT7b mediates macrophage-induced programmed cell death in patterning of the vasculature. Nature 437 (7057), 417-421 (2005).
⁵ Rao, S. et al., Obligatory participation of macrophages in an angiopoietin 2-mediated cell death switch. Development 134 (24), 4449-4458 (2007).
⁶ Tian, N. & Copenhagen, D. R., Visual stimulation is required for refinement of ON and OFF pathways in postnatal retina. Neuron 39 (1), 85-96 (2003).
⁷ Johnson, J. et al., Melanopsin-dependent light avoidance in neonatal mice. Proc Natl Acad Sci USA 107 (40), 17374-17378 (2010).
⁸ Tarttelin, E. E. et al., Expression of opsin genes early in ocular development of humans and mice. Exp Eye Res 76 (3), 393-396 (2003).
⁹ Hattar, S. et al., Melanopsin and rod-cone photoreceptive systems account for all major accessory visual functions in mice. Nature 424 (6944), 76-81 (2003).
¹⁰ Panda, S. et al., Melanopsin (Opn4) requirement for normal light-induced circadian phase shifting. Science 298 (5601), 2213-2216 (2002).
¹¹ Hattar, S., Liao, H. W., Takao, M., Berson, D. M., & Yau, K. W., Melanopsin-containing retinal ganglion cells: architecture, projections, and intrinsic photosensitivity. Science 295 (5557), 1065-1070 (2002).
¹² Graham, D., Melanopsin Ganglion Cells: A Bit of Fly in the Mammalian Eye. (1995).
¹³ Fu, Y. et al., Intrinsically photosensitive retinal ganglion cells detect light with a vitamin A-based photopigment, melanopsin. Proc Natl Acad Sci USA 102 (29), 10339-10344 (2005).
¹⁴ Ferrara, N., Gerber, H. P., & LeCouter, J., The biology of VEGF and its receptors. Nat Med 9 (6), 669-676 (2003).
¹⁵ Gariano, R. F., Hu, D., & Helms, J., Expression of angiogenesis-related genes during retinal development. Gene Expr Patterns 6 (2), 187-192 (2006).
¹⁶ Meeson, A. P., Argilla, M., Ko, K., Witte, L., & Lang, R. A., VEGF deprivation-induced apoptosis is a component of programmed capillary regression. Development 126 (7), 1407-1415 (1999).
¹⁷ Adamis, A. P. et al., Increased vascular endothelial growth factor levels in the vitreous of eyes with proliferative diabetic retinopathy. Am J Ophthalmol 118 (4), 445-450 (1994).
¹⁸ Kendall, R. L. & Thomas, K. A., Inhibition of vascular endothelial cell growth factor activity by an endogenously encoded soluble receptor. Proc Natl Acad Sci USA 90 (22), 10705-10709 (1993).
¹⁹ Shibuya, M., Structure and dual function of vascular endothelial growth factor receptor-1 (Flt-1). The international journal of biochemistry & cell biology 33 (4), 409-420 (2001).
²⁰ Chappell, J. C., Taylor, S. M., Ferrara, N., & Bautch, V. L., Local guidance of emerging vessel sprouts requires soluble Flt-1, Dev Cell 17 (3), 377-386 (2009).
²¹ Ambati, B. K. et al., Corneal avascularity is due to soluble VEGF receptor-1. Nature 443 (7114), 993-997 (2006).
²² Lichtenberger, B. M. et al., Autocrine VEGF signaling synergizes with EGFR in tumor cells to promote epithelial cancer development. Cell 140 (2), 268-279 (2010).
²³ Ashery-Padan, R., Marquardt, T., Zhou, X., & Gruss, P., Pax6 activity in the lens primordium is required for lens formation and for correct placement of a single retina in the eye. Genes Dev 14 (21), 2701-2711 (2000).
²⁴ Rowan, S. & Cepko, C. L., Genetic analysis of the homeodomain transcription factor Chx10 in the retina using a novel multifunctional BAC transgenic mouse reporter. Dev Biol 271 (2), 388-402 (2004).
²⁵ Garcia, C. M. et al, The function of VEGF-A in lens development: formation of the hyaloid capillary network and protection against transient nuclear cataracts. Exp Eye Res 88 (2), 270-276 (2009).
²⁶ Smith, L. E., Through the eyes of a child: understanding retinopathy through ROP the Friedenwald lecture. Invest Ophthalmol Vis Sci 49 (12), 5177-5182 (2008).
²⁷ Mintz-Hittner, H. A., Kennedy, K. A., & Chuang, A. Z., Efficacy of intravitreal bevacizumab for stage 3+ retinopathy of prematurity. N Engl J Med 364 (7), 603-615.
²⁸ Kennedy, K. A. et al, Reduced lighting does not improve medical outcomes in very low birth weight infants. J Pediatr 139 (4), 527-531 (2001).
²⁹ Reynolds, J. D. at al., Lack of efficacy of light reduction in preventing retinopathy of prematurity. Light Reduction in Retinopathy of Prematurity (LIGHT-ROP) Cooperative Group. N Engl J Med 338 (22), 1572-1576 (1998).
³⁰ Stockmann, C. et al., Deletion of vascular endothelial growth factor in myeloid cells accelerates tumorigenesis. Nature 456 (7223), 814-818 (2008).
   Acknowledgements We thank Paul Speeg for technical assistance. This work was supported by the NIH (D. C. and R. A. L.).
   Author Contributions R. A. L. and D. C. provided project leadership. R. A. L., D. C. and S. R. wrote the manuscript. N. F. developed critical reagents. S. R., C. C. and J. F. performed experimentation.
   Author Information Reprints and permissions information is available at npg.nature.com/reprintsandpermissions. N. F. is an employee of Genentech Corporation. Correspondence and requests for materials should be addressed to R. A. L. (richard.lang@cchmc.org).

### FIGURE LEGENDS

FIG. 1. Hyaloid vessel regression is light-dependent

(a) Hyaloid vessel preparations at the indicated postnatal (P) days from pups reared under normal light conditions (LD) or under constant darkness (DD) from E16-17. (b) As in (a) but a quantification of the number of capillaries from P1 to P8. (c) P5 quantification of the apoptotic index in hyaloid vascular cells (isolated apoptosis) or vessels undergoing a segmental pattern of apoptosis. p values as labeled, NS—not significant.

FIG. 2. Hyaloid vessel regression is dependent on the Opn4 gene product melanopsin

(a) Hyaloid vessel preparations at P3, P5 and P8 for control and Opn4^{-/-} mice. (b) P8 quantification of hyaloid capillary number in Opn4^{+/+} and Opn4^{-/-} mice. (c, d) Detection of retinal vasculature (green, isolectin labeling) and melanopsin in ipRGCs (red) in the superficial layers of the P5 mouse retina. (c) Is at 100× magnification and is located at the extending front of retinal vessels. (d) Is at 400× magnification of position peripheral to the extending vascular front. Retinal myeloid cells in (c, d) can be observed labelled at low levels with isolectin (green).

FIG. 3. sFlt1 regulates hyaloid regression but is not melanopsin-regulated

(a) P8 hyaloid vessel preparations in control Flt1^{+/+} and Flt1^{+/-} mice. (b) Quantification of hyaloid vessel numbers at P8 in control Flt1^{+/+} and Flt1^{+/-} mice. (c) P3 and P8 hyaloid vessel preparations in control Chx10-cre; Flt1^{+/+} and Chx10-cre; Flt1^{fl/fl} mice. (d) Time-course quantification of hyaloid vessel numbers in control Chx10-cre; Flt1^{+/+} (grey line) and Chx10-cre; Flt1^{fl/fl} (blue line) mice from P3 to P8. (e) Immunoblot (IB) detecting sFlt1 in the vitreous of P1, P5 and P8 mouse pups. (f) QPCR detection of sFlt1 mRNA in P5 retina from control/LD (grey bar) Opn4 mutant mice (light blue bar) and dark-reared mice (DD, dark blue bar). (g) ELISA detection of sFlt1 in P5 vitreous from control/LD mouse pups (grey bar), Opn4 mutant mice (light blue bar) and dark-reared mice (DD, dark blue bar). p values as labeled, NS—not significant.

FIG. 4. Retinal VEGF suppresses hyaloid regression and is light and melanopsin regulated.

(a) Cryosections through the vitreous of control Chx10-cre and Chx10-cre; VEGFA^{fl/fl} pups at P1. White arrowheads indicate vessels of the tunica vasculosa lentis (TVL). Yellow arrowheads indicate hyaloid vessels (HV) present only in the control. (b) P8 hyaloid vessel preparations in control VEGF^{fl/+} and Chx10-cre; VEGF^{fl/fl} mice. (c) Quantification of hyaloid vessel numbers at P8 in VEGF^{fl/+} and Chx10-cre; VEGF^{fl/fl} mice. (d) Vitreous VEGF immunoblot for wild type mice at P1, P5 and P8. A quantification of relative signal levels is shown in the histogram. (e) Immunoblot for P1 or P5 vitreous VEGF in Opn4^{+/+} and Opn4^{-/-} mice (left three panels) or in mice that were reared LD or DD light conditions (right most panels). (f) ELISA quantification of VEGF levels in the P5 vitreous of control/LD mouse pups (grey bar) from Opn4^{-/-} mice (pale blue bar) or those raised in constant darkness (DD, blue bar). (g) QPCR detection of VEGF mRNA in P5 retina from control/LD (grey bar) Opn4 mutant mice (light blue bar) and dark-reared mice (DD, dark blue bar). (h) P5 vitreous levels of VEGF and sFlt1 in control/LD mice (grey bar) Opn4^{-/-} mice (pale blue bar) or those raised in constant darkness (DD, blue bar) expressed as a molar ratio.

FIG. S1. Retinal VEGF immunoreactivity is reduced in VEGF conditional mutant mice.

To assess the ability of Chx10-cre to delete VEGF in the retina, we performed VEGF immunolabeling in P5 retinal cryosections from VEGF^{fl/fl}, Chx10-cre; VEGF^{+/fl} and Chx10-cre; VEGF^{fl/fl}. The sections were also labeled with antibodies to calretinin to mark amacrine cells and Hoechst 33258 to mark nuclei and thus locate the layers of the retina. The level of VEGF immunoreactivity is successively reduced with heterozygous and homozygous conditional deletion as would be expected.

1. A method of treating ROP, the method comprising
a. providing a light source emitting light with a wavelength of about 490 nm,
b. exposing an infant's eye to the light, and
c. monitoring the vascularization in the infant's eye.

2. The method according to claim 1, further comprising the step of ceasing the exposure when the monitoring demonstrates that vascularization has been suppressed.

3. The method according to claim 1, further comprising the step of ceasing the exposure when the monitoring demonstrates that the vascularization is regressing.

4. The method according to claim 1, further comprising optimizing the transition time.

5. The method according to claim 1, further comprising optimizing the light intensity.

6. A method for in utero light therapy comprising
a. assessing a risk of a premature birth,
b. determining a course of treatment to prevent ROP,
c. providing a light source emitting light with a wavelength of about 490 nm, and
d. securing the light source to a body, wherein the light is directed toward a fetus in the body.

7. A method of treating a vascular disease comprising:
a. providing a light source emitting light having a wavelength of about 490 nm,
b. exposing the vascularly diseased area to the light, and
c. monitoring treatment of the vascular disease against a standard.

8. A device for providing in utero light therapy comprising
a. a light source, wherein the light source provides light having a wavelength of about 490 nm, and
b. a securing device for securing the light source to a wearer's body, wherein the light is directed toward the body and wherein the light source is not directed at the eyes of the wearer.

9. The device according to claim 8, wherein the 85% of the light provided by the light source has a wavelength between 460 nn and 520 nm.

10. A device for treatment of ROP comprising
a. an incubator with a lid, and
b. a light source positioned above the lid, wherein the light provided by the light source has a wavelength of about 490 nm.

11. A device for treatment of ROP comprising
a. an incubator with a lid, and
b. a light filtering device associated with the lid, wherein the light filtering device allows passage of only light with a wavelength of about 490 nm.

12. The device according to claim 11 wherein the light filtering device is integral with the lid.

13. The device according to claim 11 wherein the light filtering device is placed on a surface of the lid.

14. A method of creating a causative stimulus for organism development comprising
a. providing a light source
b. regulating the light source, and
c. monitoring the organism development.

15. The method according to claim 14, where in the regulating step comprises regulating the exposure time of the organism to the light source.

16. The method according to claim 14, wherein the regulating step comprises regulating the wavelength of light provided by the light source.

17. The method according to claim 14, wherein the regulating step comprises regulating the intensity of light provided by the light source.

18. The method according to claim 15, wherein the regulating step further comprises regulating the wavelength of light provided by the light source.

19. The device according to claim 10, wherein the light provided by the light source has a wavelength between 460 nm and 520 nm.

20. The device according to claim 11, wherein the light filtering device allows passage of only light with a wavelength between 460 nm and 520 nm.
